(19) United States
(12) Patent Application Publication
(10) Pub. No.: US 2013/0323208 A1
(43) Pub. Date: Dec. 5, 2013

(54) MYCOBACTERIUM BRUMAE CELL WALL EXTRACTS THAT CAN BE USED IN THERAPY OF SUPERFICIAL BLADDER CANCER

(71) Applicants: Naciye Leyla Açan, Ankara (TR); Abdurrahim Haluk Özen, Ankara (TR); Esra Büber, Ankara (JP); Zühtü Tanil Kocagöz, Istanbul (TR); Zeliha Ertürk, Ankara (TR); Zehra Stara Yüksel, Instanbul (TR); Mehmet Selçuk Keskin, Instanbul (TR); Bagnu Orhan, Kirsehir (TR); Alpaslan Alp, Ankara (TR); Zeynep Saribas, Ankara (TR)

(72) Inventors: Naciye Leyla Açan, Ankara (TR); Abdurrahim Haluk Özen, Ankara (TR); Esra Büber, Ankara (JP); Zühtü Tanil Kocagöz, Istanbul (TR); Zeliha Ertürk, Ankara (TR); Zehra Stara Yüksel, Instanbul (TR); Mehmet Selçuk Keskin, Instanbul (TR); Bagnu Orhan, Kirsehir (TR); Alpaslan Alp, Ankara (TR); Zeynep Saribas, Ankara (TR)

(21) Appl. No.: 13/636,695

(22) PCT Filed: Dec. 20, 2011
(86) PCT No.: PCT/TR2011/000277

§ 371 Date: Sep. 23, 2012

(30) Foreign Application Priority Data
Feb. 25, 2011 (TR) 2011/01874

(51) Int. Cl. A61K 35/74

(52) U.S. Cl. CPC A61K 35/74
USPC 424/93.4; 424/780; 435/253.1; 435/6.15; 435/7.92

(57) ABSTRACT

Invention is using M. brumae is a mycobacterium strain which stimulates immune system cells strongly, is non-pathogen for humans and has all of the defining attributes of the strain with catalog number 51384, American Type Culture Collection (ATCC) for therapy of superficial bladder cancers.

### RELATED TECHNICAL FIELD

The invention is related to a preparation of a composition of Mycobacterium cells and cell wall extracts that can be used in therapy of superficial bladder cancer instead of Bacillus Calmette-Guerin (BCG) cells.

### STATE OF THE ART RELATED TO THE INVENTION

In therapy of superficial bladder cancer there are different therapy alternatives according to pathologic stage and grade of the disease. Cystoscopy and TUR alone are sufficient in low grade patients without Lamina propria invasion. BCG (M. bovis vaccine strain) instillation in bladder is still the standard therapy in high grade bladder cancers. Despite the therapy a large number of these patients progress to muscle invasive disease. The therapy in this stage is cystectomy in which the whole bladder is removed. Thus the therapy of high grade tumors with an immunotherapy method more effective than BCG will avoid the progress of stage. The necessity of long-term use of BCG therapy increases the risk of complications with pathogenic BCG.

There is not yet any other immunotherapy or chemotherapy method as effective as BCG.

In the patent application with publication number RU2112543 (C1) in this topic, an immunomodulatory drug is described and it proposes a preparation that is a modified BCG. Preparation is a specific immunomodulating agent containing extract from tuberculosis mycobacteria of vaccine strain BCG complexed with polyoxydonium-synthetic immunostimulating agent taken at ratio (1:30)-(1:120). Combination of this preparation with conventional antituberculosis chemotherapy enhances effectiveness of the therapy.

The patent application with publication number U.S. Pat. No. 5,759,554 (A) described the immunostimulating ability of the cell wall fraction of bacteria. This invention relates to an immunotherapeutic agent that is effective in treating a variety of diseases in animals and humans. More particularly, the present invention is a preparation of a modified mycobacterial cell wall extract that does not contain oil, that is capable of stimulating the immune system of an animal or human in such a way as to cause the body to neutralize, abort or eliminate infections, tumors and other disorders.

The patent application with publication number NZ506406 (A) describes a composition comprising Mycobacterium DNA (B-DNA) and a pharmaceutically acceptable carrier in the preparation of a medicament, wherein the medicament has anti-bladder cancer activity in an animal having bladder cancer, further wherein the B-DNA is not Mycobacterium bovis DNA.

The patent application with publication number U.S. Pat. No. 5,712,123 (A) describes a purified hot-water extract from Mycobacterium bovis (BCG vaccine). It has been found to have significant antitumor activity against a mouse bladder tumor model and a murine sarcoma in vivo, but not in vitro. The material, termed PS1A1, has an approximate molecular weight of between 60 and 90 kDa, is freely soluble in water, but has low solubility in acetone or ethanol, and is remarkably heat-stable.

In the patent application with publication number CN1339583 (A) a kind of microbial preparation, recombined smegmatism mycobacterium comprising smegmatis mycobacterium and mycobacterium shuttle expression vector, for treating bladder tumor. The mycobacterium shuttle expression vector consists of mycobacterium expressing plasmid, human tubercle bacillus heat shock protein 70 promoter cDNA, Kansas mycobacterium alpha antigen signal peptide cDNA and human interleukin-2 cDNA. The recombined smegamtis mycobacterium is used mainly for treating superficial bladder tumor in situ cancer, late stage tumor incapable of being operationtreated, preventing postoperational relapse of bladder tumor, etc. The present invention is diluted normal saline before being perfused into patient's ladder or locally injected during the treatment.

In the current invention the extracts from M. brumae grown in liquid broth are obtained by rupturing the cell wall through sonication and by separation of the supernatant through centrifugation after the cells are inactivated by heat. The stimulating effect of this invention on the cytokine secretion from human macrophage cells is higher than or equivalent to BCG. Their production in large scale does not create an important problem for environment, because they are non-pathogen rapidly-growing mycobacteria. In contrast to BCG, negligible side effects are expected, because during preparation they are inactivated by heat. M. brumae is a mycobacterial strain which does not generate disease in humans. In the therapy of bladder cancer with BCG live bacteria are given into the bladder. But these bacteria can sometimes spread to the body through blood and lead to serious illnesses. Even if M. brumae is given alive, this kind of problem does not occur, because it is not pathogen for humans. M. brumae cells can be used in therapy, because live M. brumae cells can stimulate the immune system better than cell extracts do. M. bovis BCG strain can be produced in culture medium in 3 weeks. Compared with BCG it is easier to prepare large amounts of drug from M. brumae, because it can be produced in a few days.

Technical Problems Aimed with the Present Invention

Bacillus Calmette-Guerin (BCG) cells are used in therapy of some bladder tumors as gold standard therapy following endoscopic resection. BCG leads to the release of cytokines from macrophages and stimulates the immune system. Although BCG is an effective therapeutic, the most important factor that limits its use is the pathogenesis and side effect profile that even causes to death.

For this reason more effective and less toxic agents are being investigated. The most important of these studies are made with M. phlei. The cell wall extracts of M. phlei is sufficient to show antitumor activity. In addition, a direct apoptotic effect on tumor cells is also known.

It has been shown that the cell wall extracts of some strains of non-pathogenic mycobacteria other than M. phlei have immunostimulating effect which is equal to or higher than M. phlei's and some of these strains have more cytotoxic activity on bladder tumor cells than M. phlei has.

The current invention aims to use a mycobacterial strain which is nonpathogenic, less harmful and the extracts of it is easier to prepare, in therapy of bladder cancer.

### DESCRIPTION OF THE INVENTION

This invention relates to the preparation of M. brumae and its cell wall extracts that will be used in therapy of superficial bladder cancer. The immune system stimulation characteristics of M. brumae, the developed products and description of the method applied for obtaining the product are below.

Obtaining of Pure Mycobacterial Colonies:

To obtain pure colonies of mycobacteria mycobacterial stocks are inoculated in biosafety cabinet into the Löwenstein Jensen medium. They are incubated at 37° C., until they could be seen on the surface of culture medium. For incubation a few loops of mycobacterial colony are inoculated into 50 ml-centrifuge tubes which contain 10 ml Middlebrook liquid broth (with OADC: 0.47 g/dL, 10% Oleic acid, Dextrose, Catalase). They are incubated for 2-5 days shaking at 37° C. with a speed 110 rotation/minute (KENT, 1985; WINN, 2005).

Cell Extraction:

Tubes which have been already incubated and reached to sufficient turbidity are centrifuged for 15 minutes at 4° C. at 4000 rpm. After the supernatant is removed, 30 ml phosphate buffer is added in tubes and they are vortexed. Tubes which are washed with phosphate buffer are again centrifuged for 15 minutes at 4° C. at 4000 rpm, supernatant is removed, 1 ml phosphate buffer is added in tubes and they are vortexed.

Mycobacterial cell sediments in phosphate buffer are kept for ten minutes in 95° C. water bath with the aim of killing cells and partially disintegrate the cell walls. Then the cell sediments which are cooled and put into a container filled with ice are sonicated for 3 minutes, running with 30% power making equivalent sonication of 1 minute (60 seconds) for each tube.(KENT 1985; WIN 2005) After all this phases the cell wall extract is obtained.

Protein Determination:

For detection of nucleic acid and protein absorbances were measured at 260 and 280 nm respectively. For the determination protein concentration, Bradford method was used (BRADFORD, 1976).

Investigation of the Effect of Mycobacterial Extracts on Immune Cells, Monocytes:

Monocyte Cell Culture:

Human monocyte cell line THP-1 (TSUCHIYA, 1980, TSUCHIYA, 1982) and bladder tumor cell line T-24 (RASHEED, 1977) were cultured in incubator with 5% CO₂ at 37 ° C. and in RPMI-1640 culture medium, which contains 10% fetal bovine serum inactivated with heat, 100 U/ml penicillin and 100 pg/ml streptomycin. Cells were grown with passages every 3-4 days. Number of cells was determined by microscopic examination using Thoma slide and trypan blue dye (YÜKSEL, 2009).

TNF-α and IL-12 Measurement:

For measurements Invitrogen ELISA kit protocol (Biosource) was applied. As positive control 1 μg/ml LPS was used (MARTHA, 2001). In 24-well plates 50 μl standard dilution buffer was put into standard and sample wells and 100 μl in blank well. 100 μl standard and sample solutions were added to appropriate wells. Plates were covered and kept at room temperature for 2 hours, at the end of this period supernatant in wells were removed and wells were washed 4 times. Except chromogen blank 100 μl anti-TNF-alpha with biotin (or IL-12) was added to all wells and then plate was shaked. After they had been covered and kept at room temperature for an hour, supernatant was removed and wells were washed 4 times. Stabilized chromogen was added to each well, they were kept at room temperature for 30 minutes in dark and their absorbance in 450 nm was measured by ELISA reader. Statistics was made with Minitab software package (YÜKSEL, 2010). The results for M. bovis and M. brumae are in Table 1.

TABLE 1
Cytokine activation of mycobacterial cell extracts

| | IL12 | | | TNF-alpha | | | |
|---|---|---|---|---|---|---|---|
| | (pg/μg protein) | | | (pg/μg protein) | | | |
| Strain | Mean | SD | p | Mean | SD | P < 0.05 | Pathogen |
| M. bovis (BCG) | 18.69 | 6.79 | | 3271 | 1549 | | yes |
| M. brumae | 28.37 | 7.81 | p > 0.05 | 7259 | 264 | * | no |

A large number of mycobacterial species were tested in terms of the stimulation of immune cells. By considering the immune stimulating activity, non-pathogenic property and rapid growth in culture mediums, M. brumae was selected as a candidate which has anti-tumor effect, produced in a large scale and used for 3-(4,5-Dimethylthiazol-2-yl)-2,5-diphenyltetrazolium bromide (MTT) test.

MTT Test

T-24 cells were inoculated into 96-well microplates in the form of 5000 cells/well and incubated at 37° C. in 5% CO2 for 24 hours. Mycobacterial extracts were applied to the wells at determined concentrations. At the end of 72 hours MTT reagent was added to each well so that the final concentration of each well was 5 mg/ml. At the end of the 4-hour incubation 10% SDS was added to the wells and wells were incubated 16 hours more. The results were evaluated in microplate reader at a wavelength of 570 nm (TWENTYMAN, 1987).

M. brumae was determined as a mycobacterial strains which could be effective in therapy of bladder cancer. IC50 values of M. brumae and M. phlei are shown in Table 2. Because M. phlei is a non-pathogenic strain and a candidate to be used instead of M. bovis, MTT test results were compared with M. phlei instead of M. bovis.

TABLE 2
MTT, IL-12 and TNF-alpha results of selected strains

| | MTT | | IL-12 | | TNFα | |
|---|---|---|---|---|---|---|
| | (IC₅₀, μg/ml) | | (pg/μg protein) | | (pg/μg protein) | |
| Strain | Mean | SD | Mean | SD | Mean | SD |
| M. phlei | 24.06 | 23.31 | 7.58 | 0.97 | 9908 | 699 |
| M. brumae | 14.11 | 13.24 | 28.37 | 7.81 | 7259 | 264 |

M. brumae strains were produced in Löwenstein Jensen medium, centrifuged in 2000×g for 15 minutes after incubation for 2-5 days in Middlebrook liquid medium (0.47 g/dL, 10% OADC) and the sediment was washed with 50 mM potassium phosphate buffer pH 7.0. It was kept in 95° C. water bath for 10 minutes and cooled. After sonication (15 minutes, pulse: 20 40, temp: 30° C., Amp: 40%) and centrifugation in 27000×g supernatant was used.

This sample was further separated by HPLC using C18 and SCX columns and the molecular weights of obtained fractions were analyzed by mass spectroscopy. It was found that immunostimulant activity was associated with structures whose molecular weights are between 1800 kDa and 3000 kDa.

The current invention shows that M. brumae can be used in therapy of bladder cancers. For this purpose direct live M. brumae cells or cell extracts can be used. This invention is isolates obtained by separation of cell extracts prepared from M. brumae using various chromatographic techniques. Mycobacterial strains described here is the culture filtrate of M. brumae and the isolates obtained by separation of culture filtrate. The M. brumae strain here is registered with American Type Culture Collection (ATCC) catalog No: 51384, yellow colored, “nonphotochromogenic” colony forming, non-pathogenic, rapidly-growing strain (LUQUIN, 1993).

M. brumae strain here can be used in combination with other substances to manufacture a pharmaceutical composition or formulation. M. brumae strain is proposed currently for treatment since it is not pathogenic and less harmful as compared to BCG strains. In addition because of the rapid growth of M. brumae in culture medium it is easier to prepare preparations produced by using M. brumae. This invention was discovered within the context of Project SBAG-SANTEZ-5-105S361 supported by Ministry of Industry and Commerce through TUBITAK.

The above mentioned preferred applications have been defined so that the present invention is better understood, and that they are not of limiting nature for the scope of protection. The preferred applications, the changes to be made in accordance with the information disclosed with the invention must be considered to be within the scope of protection of the invention.

### REFERENCES

BRADFORD M. M., A rapid and sensitive method for the quantitation of microgram quantities of protein utilizing the principle of protein dye binding, Anal Biochem, 72, 248-54, (1976).

CHIMARA E., Ferrazoli L., Ueki S. Y., Martins M. C., Durham A. M., Arbeit R. D., Leao S. C., Reliable identification of mycobacterial species by PCR-restriction enzyme analysis (PRA)-hsp65 in a reference laboratory and elaboration of a sequence-based extended algorithm of PRA-hsp65 patterns, BMC Microbiol 8: 48 (2008).

KENT P. T., Kubica G. P., Public Health Mycobacteriology, A Guide for the Level III Laboratory. US Department of Health and Human Services, Atlanta, (1985). Pp 5-12. LUQUIN M., et al. Mycobacterium brumae sp. nov., a rapidly growing, nonphotochromogenic mycobacterium. Int. J. Syst. Bacteriol. 43, 405-413, (1993).

MARTHA M., Oliveira R. C., Cristina V., Mycobacterium bovis BCG but not Mycobacterium leprae induces TNF-a secretion in human monocytic THP-1 cells, Mem Inst Oswaldo Cruz, Rio de Janeiro, 96, 973-978, (2001).

RASHEED S., Gardner M. B., Rongey R. W., Nelson-Rees W. A., Arnstein P., Human bladder carcinoma: characterization of two new tumor cell lines and search for tumor viruses, J Nat Cancer Inst, 58, 881-890, (1977).

TSUCHIYA S., Yamabe M., Yamaguchi Y., Kobayashi Y., Konno T., Tada K., Establishment and characterization of a human acute monocytic leukemia cell line (THP-1), Int J Cancer, 26(2), 171-6, (1980).

TSUCHIYA S., Kobayashi Y., Goto Y., Okumura H., Nakae S., Konno T., Tada .K., Induction of maturation in cultured human monocytic leukemia cells by a phorbol diester, Cancer Res, 42(4), 1530-6, (1982).

TWENTYMAN P. R., Luscombe M., A study of some variables in a tetrazolium dye (MTT) based assay for cell growth and chemosensitivity, Br J Cancer. 56, 279-285 (1987).

WINN JR W., Allen S., Janda W., Koneman E., Procop G., Schreckenberger P., Woods G. Konemann's Color Atlas and Textbook of Diagnostic Microbiology, Lippincott William and Wilkins, Philadelphia, (2005).

YÜKSEL, Z. S., Identification of the mycobacterial species that stimulate the immune system most efficiently for the treatment of bladder cancer, (MSc Thesis) Marmara University, Faculty of Engineering, Department of Bioengineering, (2009).

YUKSEL Z. S., Buber E., Kocagoz T., Alp A., Sariba [symbol] Z., Açan N. L., Mycobacterial strains that stimulate the immune system most efficiently as a candidate for the treatment of bladder cancer, J. Mol Microbiol Biotechnol, 20(1): 24-8 (2011).

1-7. (canceled)

8. A pharmaceutical composition used to treat cancer the pharmaceutical composition comprising: M. brumae strain; and any other substance.

9. A method of treating bladder cancer the method comprising: using direct live M. brumae cells or cell extracts.

10. A method of obtaining cell extracts from M. brumae the method comprising: separating cell extracts from M. brumae by using chromatographic techniques.

11. A method of obtaining pure mycobacterial colonies the method comprising: inoculating stocks at 37° C. in a Löwenstein Jensen medium until they could be seen on the culture medium; inoculating a few loops of mycobacterial colony in a 50 ml-centrifuge tubes wherein tubes contain 10 ml Middlebrook liquid broth, OADC: 0.47 g/dL, 10% Oleic acid, Dextrose, Catalase; rotating and shaking said tubes for 2 to 5 days with rotation speed of 100 rotation/minute at temperature 37° C.

12. The method of claim 11 the method further comprising: centrifuging tubes that reached to a sufficient turbidity for 15 minutes at 4° C. at 4000 rpm; observing to see if the supernatant is separated; adding 30 ml phosphate buffer in tubes upon the removal of the supernatant; vortexing tubes; centrifuging tubes for 15 minutes at 4° C. at 4000 rpm, observing to see that the supernatant is removed; adding 1 ml phosphate buffer in tubes upon removing the supernatant; and vortexing the tubes.

13. The method of claim 12 the method further comprising: keeping mycobacterial cell sediments in phosphate buffer for about ten minutes in water with temperature of 95 ° C. bath; placing cell sediments in a container for about 3 minutes wherein the container is filled with ice; running with 30% power making equivalent sonication of 1 minute (60 seconds) for each tube.

14. A method for detecting protein absorbance of the solution of claim 13 or detecting nucleic acid in the solution of claim 13 the method comprising: using bradford method.

15. A method of measuring effect of mycobacterial extracts on immune cells the method comprising: culturing human monocyte cell line THP-1 and bladder tumor cell line T-24 in an incubator with 5% CO2 at 37 ° C. and in RPMI-1640 culture medium which contains 10% fetal bovine serum inactivated with heat, 100 U/ml penicillin and 100 μg/ml streptomycin; keeping this set up for at least 3 to 4 days; and counting number of cells by microscopic examination using Thoma slide and trypan blue dye.

16. A method of measuring TNF-α and IL-12 the method comprising: using positive control 1 μg/ml LPS (MARTHA, 2001); placing 50 μl standard dilution buffer into standard and sample wells and placing 100 μl in blank well; adding 100 μl standard and sample solutions to appropriate wells; covering plates; keeping plates at room temperature for 2 hours; removing supernatant in wells; washing wells about four times; adding chromogen blank 100 μl anti-TNF-alpha with biotin (or IL-12) to all wells; shaking wells; keeping wells at room temperature for an hour; removing supernatant; washing wells about four times; adding stabilized chromogen to each well; keeping wells at room temperature for 30 minutes in dark; and measuring their absorbance in 450 nm by ELISA reader.

17. A method of measuring MTT the method comprising: inoculating T-24 cells into plurality of 96-well microplates in the form of 5000 cells/well; incubating the plurality of 96-well microplates at 37° C. in 5% CO2 for about 24 hours; applying mycobacterial extracts to the plurality of wells at pre-determined concentrations; adding the MTT reagent to each well at the end of 72 hours so that the final concentration of each well is 5 mg/ml; adding 10 percent SDS to the plurality of wells at the end of the four hours incubation period; incubating wells for additional sixteen hours; reading results in microplate reader at a wavelength of 570 nm.
